(19) United States
(12) Patent Application Publication
(10) Pub. No.: US 2022/0284269 A1
(43) Pub. Date: Sep. 8, 2022

(54) SYSTEM FOR CONTROL AND ANALYSIS OF GAS FERMENTATION PROCESSES

(71) Applicant: LanzaTech, Inc., Skokie, IL (US)

(72) Inventors: James Howden Daniell, Auckland (NZ); Steven Samuel Glasker, Auckland (NZ); Michael Emerson Martin, Chicago, IL (US); Melvin Moore, Chicago, IL (US); Michael James Harry Mawdsley, Wellington (NZ); Jim Jeffrey Daleiden, Niles, IL (US)

(21) Appl. No.: 17/653,077

(22) Filed: Mar. 1, 2022

Related U.S. Application Data
(60) Provisional application No. 63/156,241, filed on Mar. 3, 2021.

(51) Int. Cl. G06N 3/04; G06K 9/62; C12P 7/06

(52) U.S. Cl. CPC G06N 3/0454; G06K 9/6256; C12P 7/06

(57) ABSTRACT

This disclosure relates to analyzing a fermentation process that occurs in a bioreactor. Such a fermentation process may involve microbes consuming a substrate, and producing various metabolites. A computing device may train and execute one or more machine learning models to analyze such a fermentation process. Such a machine learning model may be configured to determine a current fermentation state of such a fermentation process as one example. As another example, a machine learning model may be configured to predict metabolite production of a fermentation process based on historical fermentation data and a window of control decisions for the fermentation process.

### CROSS REFERENCE TO RELATED APPLICATIONS

This application claims the benefit of U.S. Provisional Patent Application No. 63/156,241, filed Mar. 3, 2021, the entirety of which is incorporated herein by reference.

### FIELD

This application relates to analysis and control of gas fermentation processes.

### BACKGROUND

Mitigation of impending climate change requires drastic reductions in emissions of greenhouse gases (GHGs), such as those generated through the burning of fossil fuels like coal and oil. Although sustainable sources of fuels and chemicals are currently insufficient to significantly displace our dependence on fossil carbon, gas fermentation has recently emerged as an alternative platform for the biological fixation of such gases such as CO, CO₂, and/or H₂ into sustainable fuels and chemicals. In particular, gas fermentation technology can utilize a wide range of feedstocks including gasified carbonaceous matter (e.g., municipal solid waste or agricultural waste) or industrial waste gases (e.g., from steel mills or oil refineries) to produce ethanol, jet fuel, and a variety of other products. Gas fermentation alone could displace 30% of crude oil use and reduce global CO₂ emissions by 10%, but, as with any disruptive technology, many technical challenges must be overcome before this potential is fully achieved. For instance, the behavior of such gas fermentation processes may be difficult to understand, manage, and predict.

### DESCRIPTION OF THE DRAWINGS

FIG. 1 shows an example fermentation system that is configured with one or more of examples of the techniques as disclosed herein;

FIG. 2 shows an example computing device in accordance with one or more aspects described herein;

FIG. 3 shows an example conceptual diagram of a machine learning model in accordance with one or more aspects described herein;

FIG. 4 shows an example conceptual diagram of a machine learning model in accordance with one or more aspects described herein;

FIG. 5 shows an example conceptual diagram of a machine learning model in accordance with one or more aspects described herein;

FIG. 6 illustrates an example flow chart of a process for configuring a model to determine a fermentation state;

FIG. 7 illustrates an example flow chart of a process for configuring a model to determine such a fermentation state; and

FIG. 8 illustrates an example flow chart of a process for configuring a model to determine such a fermentation state.

### DETAILED DESCRIPTION

Unless otherwise defined, the following terms as used throughout this specification are defined as follows:

The terms “increasing the efficiency,” “increased efficiency,” and the like, when used in relation to a fermentation process, include, but are not limited to, increasing one or more of the rate of growth of microorganisms catalyzing the fermentation, the growth and/or product production rate at elevated product concentrations, increasing the volume of desired product produced per volume of substrate consumed, increasing the rate of production or level of production of the desired product, increasing the relative proportion of the desired product produced compared with other by-products of the fermentation, decreasing the amount of water consumed by the process, and decreasing the amount of energy utilized by the process.

The term “fermentation” should be interpreted as a metabolic process that produces chemical changes in a substrate. For example, a fermentation process receives one or more substrates and produces one or more products through utilization of one or more microorganisms. The term “fermentation,” “gas fermentation” and the like should be interpreted as the process which receives one or more substrate, such as syngas produced by gasification and produces one or more product through the utilization of one or more C1-fixing microorganism. The fermentation process may include the use of one or more bioreactor. The fermentation process may be described as either “batch” or “continuous”. “Batch fermentation” is used to describe a fermentation process where the bioreactor is filled with raw material, e.g. the carbon source, along with microorganisms, where the products remain in the bioreactor until fermentation is completed. In a “batch” process, after fermentation is completed, the products are extracted, and the bioreactor is cleaned before the next “batch” is started. “Continuous fermentation” is used to describe a fermentation process where the fermentation process is extended for longer periods of time, and product and/or metabolite is extracted during fermentation. The fermentation process may be continuous.

The term “non-naturally occurring” when used in reference to a microorganism is intended to mean that the microorganism has at least one genetic modification not found in a naturally occurring strain of the referenced species, including wild-type strains of the referenced species. Non-naturally occurring microorganisms are typically developed in a laboratory or research facility.

The terms “genetic modification,” “genetic alteration,” or “genetic engineering” broadly refer to manipulation of the genome or nucleic acids of a microorganism by the hand of man. Likewise, the terms “genetically modified,” “genetically altered,” or “genetically engineered” refers to a microorganism containing such a genetic modification, genetic alteration, or genetic engineering. These terms may be used to differentiate a lab-generated microorganism from a naturally-occurring microorganism. Methods of genetic modification of include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization.

The terms “polynucleotide,” “nucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides or nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene products.”

The terms “polypeptide”, “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

“Enzyme activity,” or simply “activity,” refers broadly to enzymatic activity, including, but not limited, to the activity of an enzyme, the amount of an enzyme, or the availability of an enzyme to catalyze a reaction. Accordingly, “increasing” enzyme activity includes increasing the activity of an enzyme, increasing the amount of an enzyme, or increasing the availability of an enzyme to catalyze a reaction. Similarly, “decreasing” enzyme activity includes decreasing the activity of an enzyme, decreasing the amount of an enzyme, or decreasing the availability of an enzyme to catalyze a reaction.

A “microorganism” is a microscopic organism, especially a bacterium, archaeon, virus, or fungus. The microorganism used is typically a bacterium. As used herein, recitation of “microorganism” should be taken to encompass “bacterium.”

“C1” refers to a one-carbon molecule, for example, CO, CO₂, CH₄, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for the microorganism. For example, a C1-carbon source may comprise one or more of CO, CO₂, CH₄, CH₃OH, or CH₂O₂. The C1-carbon source may comprises one or both of CO and CO₂. A “C1-fixing microorganism” is a microorganism that has the ability to produce one or more products from a C1 carbon source. Typically, the microorganism is a C1-fixing bacterium.

An “anaerobe” is a microorganism that does not require oxygen for growth. An anaerobe may react negatively or even die if oxygen is present above a certain threshold. However, some anaerobes are capable of tolerating low levels of oxygen (e.g., 0.000001-5% oxygen). Typically, the microorganism may be an anaerobe according to some examples.

An “ethanologen” is a microorganism that produces or is capable of producing ethanol. Microorganisms described herein may be ethanologens.

An “autotroph” is a microorganism capable of growing in the absence of organic carbon. Instead, autotrophs use inorganic carbon sources, such as CO and/or CO₂. Typically, the microorganisms described herein may be autotrophs.

A “carboxydotroph” is a microorganism capable of utilizing CO as a sole source of carbon and energy. Typically, the microorganisms described herein may be carboxydotrophs.

A “methanotroph” is a microorganism capable of utilizing methane as a sole source of carbon and energy. In certain embodiments, the microorganism is a methanotroph or is derived from a methanotroph. In other embodiments, the microorganism is not a methanotroph or is not derived from a methanotroph.

The term “constituents”, “contaminants”, and the like, as used herein, refers to the microbe inhibitors, and/or catalyst inhibitors that may be found in the gas stream. In particular embodiments, the constituents include, but are not limited to, sulphur compounds, aromatic compounds, alkynes, alkenes, alkanes, olefins, nitrogen compounds, phosphorous-containing compounds, particulate matter, solids, oxygen, halogenated compounds, silicon-containing compounds, carbonyls, metals, alcohols, esters, ketones, peroxides, aldehydes, ethers, and tars.

The term “treated gas”, “treated stream” and the like refers to the gas stream that has been passed through at least one removal module and has had one or more constituent removed and/or converted.

In certain embodiments, the fermentation is performed in the absence of carbohydrate substrates, such as sugar, starch, lignin, cellulose, or hemicellulose.

A microorganism may be cultured with a gas. Such culturing may be performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. The aqueous culture medium is an anaerobic microbial growth medium, such as a minimal anaerobic microbial growth medium. Suitable media are well known in the art.

The culture/fermentation should desirably be carried out under appropriate conditions for production of the target product. Typically, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting, since products may be consumed by the culture under gas-limited conditions.

Operating a bioreactor at elevated pressures allows for an increased rate of gas mass transfer from the gas phase to the liquid phase. Accordingly, the culture/fermentation may be conducted at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. However, in general, the fermentation may be operated at a pressure higher than atmospheric pressure. Also, since a given gas conversion rate is in part a function of substrate retention time and achieving a desired retention time in turn dictates the required volume of a bioreactor, the use of pressurized systems can greatly reduce the volume of the bioreactor required, and consequently the capital cost of the fermentation equipment.

In certain embodiments, the fermentation is performed in the absence of light or in the presence of an amount of light insufficient to meet the energetic requirements of photosynthetic microorganisms. In certain embodiments, the microorganism used is a non-photosynthetic microorganism.

As used herein, the terms “fermentation broth” or “broth” refer to the mixture of components in a bioreactor, which includes cells and nutrient media. As used herein, a “separator” is a module that is adapted to receive fermentation broth from a bioreactor and pass the broth through a filter to yield a “retentate” and a “permeate.” The filter may be a membrane, e.g. a cross-flow membrane or a hollow fibre membrane. The term “permeate” is used to refer to substantially soluble components of the broth that pass through the separator. The permeate will typically contain soluble fermentation products, byproducts, and nutrients. The retentate will typically contain cells. As used herein, the term “broth bleed” is used to refer to a portion of the fermentation broth that is removed from a bioreactor and not passed to a separator.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells may be recycled back to the bioreactor. The cell-free permeate remaining after target products have been removed may also be returned to the bioreactor. Additional nutrients may be added to the cell-free permeate to replenish the medium before it is returned to the bioreactor.

### EXAMPLES

The following examples further illustrate the disclosure but, of course, should not be construed to limit its scope in any way.

Chemical engineering systems may utilize a bioreactor to support a biochemical process, such as fermentation. Some fermentation processes may involve microorganisms, such as bacteria, that obtain energy and nutrients from a substrate and media in a bioreactor and as a result of a fermentation process, produce usable metabolites, such as, for example, ethanol and/or acetate.

Such a fermentation process that occurs in a bioreactor may have many variables, inputs, and outputs, all of which make a fermentation process exceedingly complex. For example, a bioreactor may be configured to receive multiple inputs, all of which may affect the fermentation process in various manners. The bioreactor system may also be configured to control outputs from the bioreactor. Such outputs may include flows of various solids and/or liquids from the bioreactor. Each of the outputs may also affect the fermentation process in various manners.

In addition to the inputs and outputs to the bioreactor, various other parameters may affect the fermentation process. For instance, the temperature, pH level, and agitation of the bioreactor may have various effects on the fermentation process. There are numerous other factors that may control a fermentation process as well.

Such fermentation processes may be exceedingly delicate. Even small changes to the process may result in termination of a fermentation process (referred to as a “run”). For example, pH level inside a bioreactor that is too high may cause the microorganisms inside the bioreactor to perish, thereby terminating a run. Starting a new run after a previous run has terminated may be time-consuming and/or expensive. On the other hand, the longer a run can be extended, the greater the amount of chemical products (e.g., metabolites) that can be produced by the microorganisms that are involved in the fermentation process. Similarly, if more metabolites can be produced per run, the more efficient, and potentially profitable, that run may be.

However, managing and analyzing biochemical processes, such as fermentation process that occur in a bioreactor, may be very difficult and labor-intensive. For example, currently, a fermentation scientist, who often has a PhD, may oversee such fermentation runs. Based on extensive experience and training, such a fermentation scientist may be able to identify a current or previous state of a fermentation process. For instance, a fermentation scientist may be able to determine whether the fermentation process is in a stable state, is improving or declining in performance, and/or is in an upset state.

Utilizing such highly-trained fermentation scientists to oversee fermentation runs is costly and labor-intensive. And, no known techniques currently exist that would allow non-fermentation experts to oversee fermentation runs, for example to determine a fermentation state of a run and/or to determine how changing parameters of a run may affect future performance of that run.

The techniques of this disclosure are directed to solving the aforementioned problems by utilizing machine learning techniques, such as deep learning techniques to generate machine learning models for analyzing a fermentation process, such as a fermentation process in a bioreactor. Such models may determine one or more characteristics associated with a fermentation process, and/or output one or more determined characteristics. As an example, such models may determine a state of the fermentation process, and output the determined fermentation state (e.g., for display). Determining and displaying a current fermentation state may allow fewer personnel and/or personnel having less technical skill than a fermentation scientist to manage a fermentation process, which in turn, may lower the costs of managing such fermentation processes, as just one advantage.

Aspects of this disclosure are also directed to using machine learning techniques, such as deep learning, to generate models that may be used to predict one or more characteristics of a fermentation process, such as a fermentation process that occurs in a bioreactor. For example, such models may be able to predict the behavior of a fermentation run for a future window of time, such as future metabolite production, and/or how changes in various control decisions may affect a fermentation process. Currently, personnel overseeing a fermentation process lack insight as to how a fermentation process will perform in the future. Accordingly, aspects of this disclosure may provide personnel overseeing a fermentation process with greater information regarding the future of such a process, which may in turn, extend the length of a fermentation run.

Additionally, such models may be configured to determine whether a problem is occurring with a fermentation process, or is likely to occur in the future. And, if a model identifies a problem and/or that a problem is likely to occur, such a model may be configured to determine one or more actions that may correct the identified problem. The model may take such corrective action on its own, and/or may output the determined actions so that personnel may evaluate and take such corrective actions. Taking such corrective actions may extend the efficiency, such as the duration of a fermentation run, which may increase metabolite output, and/or profitability of the run, as just some example advantages.

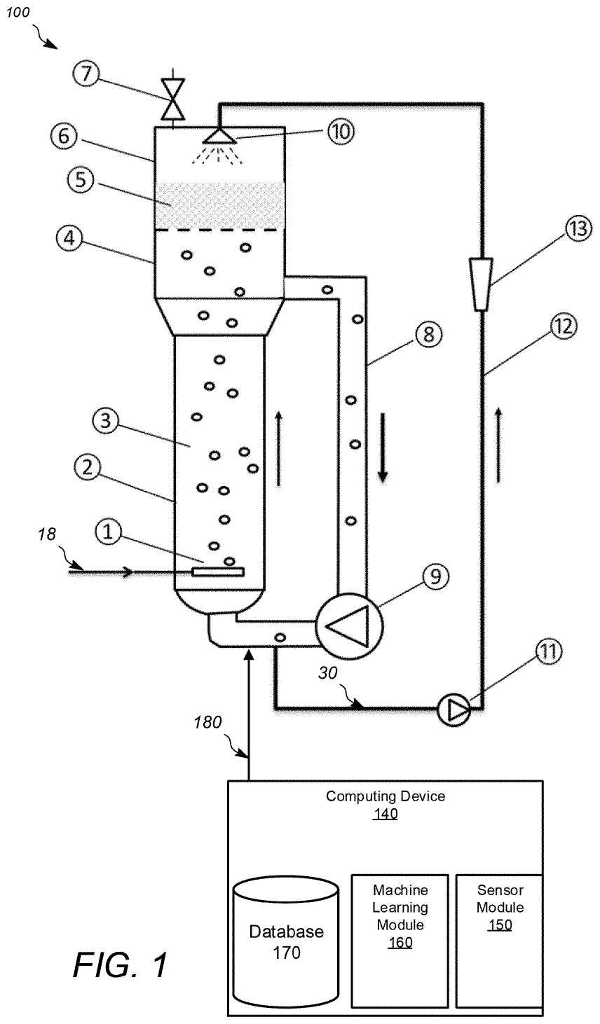

As explained above, a fermentation process may receive one or more substrates and produce one or more products through utilization of one or more microorganisms. The fermentation process may occur in a bioreactor, which may be part of system that may comprise one or more computing devices that may be configured to analyze the behavior of such a fermentation system. FIG. 1 illustrates an example of such a fermentation system comprising a bioreactor and at least one such computing device.

As explained above, fermentation processes may involve a bioreactor. FIG. 1 depicts one possible type of bioreactor 100. FIG. 1 is a schematic diagram of an embodiment of a circulated loop reactor comprising a secondary loop. The reactor consists of a riser (2), where the liquid and gas mixture (3) flow concurrently upwards, and a downcomer (8), where the liquid and gas flows concurrently downwards. The riser (2) and downcomer (8) are connected by two horizontal sections at each end and the two-phase flow is driven at least partly by a pump (9) at the bottom of the downcomer (8). The gas is introduced into the system via one or more appropriate type of spargers (1). A portion of the gas is carried over into the downcomer (8) by the liquid flow at the gas-liquid separator (4), and this portion of the gas is referred to as the “entrained gas” or the “recycled gas” herein. The non-entrained gas leaves the system via a control valve (7), after passing the headspace (6). Typically, there is a foam layer (5) of some height above the liquid level in the headspace (6). It can be seen from this diagram that the liquid and gas mixture flows from the riser (2) to the downcomer (8) and back forming a loop (i.e., the primary loop).

The secondary loop circulates fermentation broth (3) from the bottom of the downcomer (8) to the top of the reactor. Downstream of the primary loop pump (9) but before the riser (2), a stream of liquid is withdrawn from the discharge of the loop pump (9) by a secondary pump (11). The stream is passed to the top of the reactor via the secondary loop line (12), where the liquid is sprayed in the headspace (6) via an appropriate spray nozzle or shower head (10). The liquid is sprayed on to the surface of the foam layer (5) at a velocity from about 0.5 m/s to about 5 m/s in order to break up the foam. The liquid flow rate of the secondary loop is measured and monitored by a flow meter (13).

Computing device 140 may comprise a computing device that may be coupled electrically, and/or wirelessly with bioreactor 100 via at least one interface 180. For instance, computing device 140 may be coupled to one or more: sensors, displays, flow meter 13, outlet 30, inlet 18, and/or downcomer 10, as some non-limiting examples. Computing device 140 may be configured to determine or obtain measurements of such sensors, inlets, outlets, and/or other components of bioreactor 100 via communication interface 180.

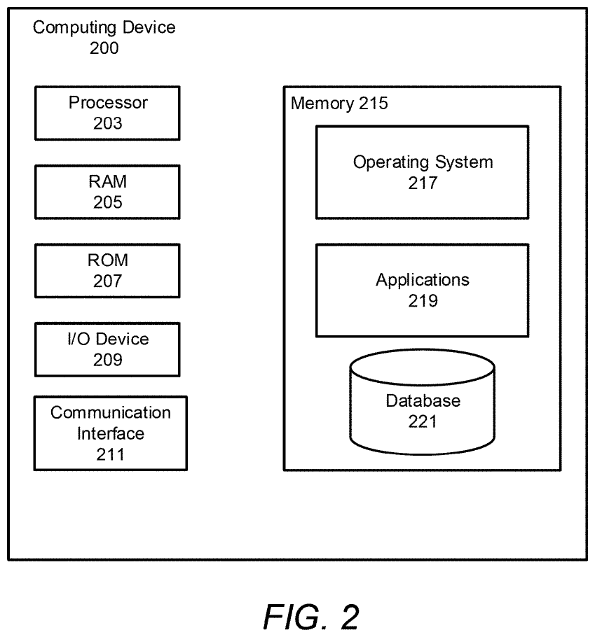

As described above, computing device 140 may be configured to define, train, and/or execute one or more machine learning models. In some examples, computing device 140 may comprise hardware that may accelerate the definition, training, and/or execution of such models. Such hardware may include one or more of: a central processing unit (CPU), application-specific integrated circuit board (ASIC), field programmable gate array (FPGA), graphics processing unit (GPU), and/or a machine learning accelerator. Such acceleration hardware may take various other forms as well. Any of the computing devices described herein may be implemented, in whole or in part, using one or more computing systems described with respect to FIG. 2.

According to some examples, computing device 140 may be configured to execute one or more of: sensor module 150, machine learning module 160, and/or database 170. Sensor module 150 may be configured to obtain sensor data from bioreactor 100, for example via communication interface 180. Machine learning module 160 may be configured to define, train, and/or execute one or more machine learning models, such as deep learning models, as just some examples. Database 170 may be configured to store and retrieve data. As examples, database 170 may be configured to store: sensor data obtained from bioreactor 100, and/or training data for a machine learning model. Database 170 may be configured to store and/or retrieve various other types of data as well.

Computing device 140 (e.g., machine learning module 160 executing on one or more processors of computing device 140) may be configured to define, train, execute, and/or configure machine learning modules in various manners. In some examples, computing device 140 may receive one or more machine learning models, which have already been configured. Such machine learning modules may be received, for example, from another computing device, such as a client computing device, as just one example.

In some examples, a computing device, such as computing device 140 may execute code in conjunction with a machine learning framework to define such a machine learning model. Examples of such frameworks may include PyTorch, TensorFlow, Keras, Apache MXNet, and/or CNTK, as just some examples. Additionally or alternatively, such machine learning models may be defined by executing code written in a program language that does not interface with such frameworks. Such machine learning models may be defined in various other manners as well.

The definition of a given machine learning model may define an architecture and/or parameters of such a model. Such an architecture may specify input and outputs of the model, as well as the types of processing operations that the model may perform. Examples of such machine learning architectures may include: support vector machines (SVMs), supervised learning algorithms, unsupervised learning algorithms, clustering algorithms, reinforcement learning algorithms, self-learning algorithms, feature learning algorithms, and/or deep learning algorithms. Machine learning models may take various other forms as well.

In some examples, such models may comprise neural networks, which may be used in deep learning models. Deep learning models may comprise one or more layers of artificial neural networks that may be connected to each other in a sequence. Such a model may take an input. Each subsequent layer of the model may extract progressively higher-level features from the input. Such layers may take various forms. For instance, a layer may comprise a convolutional neural network (CNN), recurrent neural network (RNN), such as a bi-directional long short-term memory network or gated recurrent unit (GRU), pooling layer, output layer, input layer, and/or dense/fully connected layer, as just some examples. In some examples, such layers may be combined together in a single layer. Such layers may take various other forms as well

Once a machine learning model has been defined, the model may be trained with training data. Such training data may comprise ground truth data. Such training may occur during a training phase. Based on the training data, the machine learning model may “learn” to detect various features in the input data. Based on those learned features, the model may generate one or more outputs. Training such a model may generally involve using any of various techniques to generate an output function that minimizes error of a cost function.

For a machine learning model comprised of layers of neural networks, connections between neurons of each layer may be weighted. During training, these weights may be updated to better match a prediction generated by the model, to a target output (e.g., a known state or value). Such a model may be trained until some satisfactory condition, such as a target prediction error rate, is reached.

In addition to node weights that may be derived from training, such models may have higher-level parameters, referred to as “hyperparameters,” which may not be determined as a result of training. Some examples of hyperparameters may include: a number of layers (which may include a number of hidden layers), number of neurons in a given layer, layer type, momentum, and/or learning rate, as just some examples. Model hyperparameters may be tuned by a programmer, and/or by using software, to produce more accurate models.

After a model is defined and trained, non-training (e.g., non-ground truth) data may be input into the model as part of an execution phase. Based on the inputted data, a computing device may execute the model, which may involve each layer of the model processing output from the previous layer, and finally generating output data.

As described above, one or more models as described herein may receive input data, for example, associated with bioreactor 100, and/or database 170, as some examples, and may output data that may be associated with a fermentation process. In some examples, such machine learning models may receive historical fermentation data as input. Such historical fermentation data may have been obtained from already-completed fermentation runs. Such models may output data based on the historical fermentation data. Additionally, or alternatively, such models may receive input data that is associated with an ongoing fermentation process.

Such machine learning models may receive other types of input data as well. For example, a machine learning model may receive synthetic fermentation data as input. Such synthetic fermentation data may comprise data that attempts to computationally simulate a fermentation process.

According to some examples, the techniques of this disclosure may be directed to a model that may be configured to output data associated with a state of a fermentation process that occurs in a bioreactor. Such data associated with a fermentation state may comprise, for example, a prediction of a current state of the fermentation process. For example, such a model may determine at a given time point of a fermentation occurring inside a bioreactor. Such a model may be configured to determine a fermentation state in various manners.

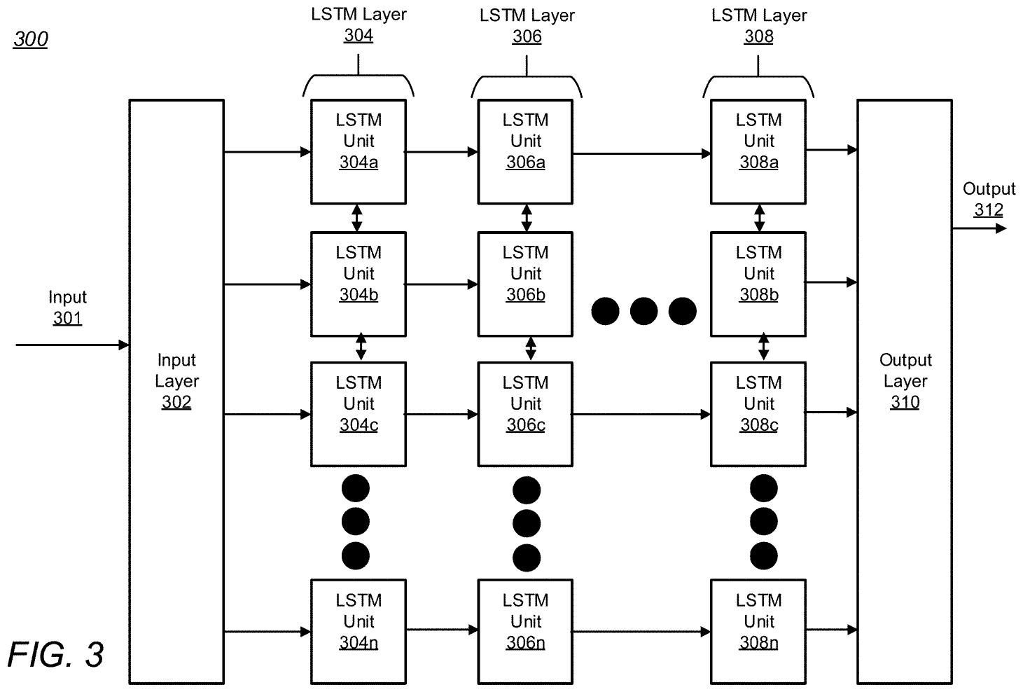

According to an example, such a fermentation state determination model may be configured as a deep learning neural network that is configured to classify a fermentation state into one of a plurality of known fermentation states. Such a model may be referred to as a “classification model,” and may be configured to determine, based on input data associated with a fermentation process, probabilities for each of the plurality of fermentation states. Such probabilities may indicate a likelihood or confidence, as determined by the model, that the fermentation process in a given fermentation state. Such a classification module is described in greater detail herein, for example with respect to FIG. 3.

According to some further examples, a fermentation state determination model may be configured to use similarity learning techniques to determine a fermentation state associated with a fermentation process. Such a similarity learning model may comprise a deep learning model that is configured to execute a similarity function. At a high level, such a similarity function may determine how similar two pieces of input data are to each other. In the context of a bioreactor fermentation process, one input to such a similarity learning model may be data associated with a known fermentation state, and another input to the similarity learning model may be fermentation data that are associated with a unknown fermentation state. By iterating through data associated with each known fermentation state, and comparing input data for each known state with the unknown state input, the model may determine a fermentation state that most closely matches the unknown fermentation state. The unknown fermentation state may then be classified as the closest-matching fermentation state. In this manner, such a similarity learning fermentation state model may be configured to determine a fermentation state of a fermentation process. Such similarity learning models are described in greater detail herein, for example with respect to FIG. 3.

Such a fermentation state that may be determined by such models may take various forms. For example, such a fermentation state may indicate: an inoculation event, stable state, fermentation performance decline, or a fermentation process upset, as just some examples. Such fermentation states may take various other forms as well.

Computing device 140 may output a fermentation state that may be output by such models. For instance, computing device 140 may be coupled to a display device, such as a monitor, display panel, touch screen, etc. Such a display device may be configured to display the current fermentation state output by computing device 140, and/or other data associated with a fermentation process. In some instances, such a display device may be located proximally to bioreactor 100 so that personnel may determine the current state of a fermentation process by viewing the display device. Additionally or alternatively, a determined fermentation state may be output to a monitoring system. Such a monitoring system may, for example, be configured to generate an alert if a determined fermentation state is in decline or indicates a process upset, as just some examples.

Turning now to FIG. 2, a conceptual illustration of a computing device 200 that may be used to perform any of the techniques as described herein is shown. The computing device 200 may include a processor 203 for controlling overall operation of the computing device 200 and its associated components, including RAM 205, ROM 207, input/output device 209, communication interface 211, and/or memory 215. A data bus may interconnect processor(s) 203, RAM 205, ROM 207, memory 215, I/O device 209, and/or communication interface 211. In some embodiments, computing device 200 may represent, be incorporated in, and/or include various devices such as a desktop computer, a computer server, a mobile device, such as a laptop computer, a tablet computer, a smart phone, any other types of mobile computing devices, and the like, and/or any other type of data processing device.

Input/output (I/O) device 209 may include a microphone, keypad, touch screen, and/or stylus through which a user of the computing device 200 may provide input, and may also include one or more of a speaker for providing audio output and a video display device for providing textual, audiovisual, and/or graphical output. Software may be stored within memory 215 to provide instructions to processor 203 allowing computing device 200 to perform various actions. Memory 215 may store software used by the computing device 200, such as an operating system 217, application programs 219, and/or an associated internal database 221. The various hardware memory units in memory 215 may include volatile and nonvolatile, removable and non-removable media implemented in any method or technology for storage of information such as computer-readable instructions, data structures, program modules, or other data. Memory 215 may include one or more physical persistent memory devices and/or one or more non-persistent memory devices. Memory 215 may include, but is not limited to, random access memory (RAM) 205, read only memory (ROM) 207, electronically erasable programmable read only memory (EEPROM), flash memory or other memory technology, optical disk storage, magnetic cassettes, magnetic tape, magnetic disk storage or other magnetic storage devices, or any other medium that may be used to store the desired information and that may be accessed by processor 203.

Communication interface 211 may include one or more transceivers, digital signal processors, and/or additional circuitry and software for communicating via any network, wired or wireless, using any protocol as described herein. It will be appreciated that the network connections shown are illustrative and any means of establishing a communications link between the computers may be used. The existence of any of various network protocols such as TCP/IP, Ethernet, FTP, HTTP and the like, and of various wireless communication technologies such as GSM, CDMA, Wi-Fi, LTE, and 5G is presumed, and the various computing devices described herein may be configured to communicate using any of these network protocols or technologies. Additionally or alternatively, communication interface 211 may comprise any of various communication interfaces, such as inter-integrated circuit (I²C), RS-232, Universal Serial Bus (USB), Ethernet, any of the Wi-Fi family of standards, supervisory control and data acquisition (SCADA), Bluetooth, and/or any other any other communication interface now known, or developed in the future.

Processor 203 may include a single central processing unit (CPU), which may be a single-core or multi-core processor, or may include multiple CPUs. Processor(s) 203 and associated components may allow the computing device 200 to execute a series of computer-readable instructions to perform some or all of the processes described herein. Although not shown in FIG. 2, various elements within memory 215 or other components in computing device 200, may include one or more caches including, but not limited to, CPU caches used by the processor 203, page caches used by the operating system 217, disk caches of a hard drive, and/or database caches used to cache content from database 221. For embodiments including a CPU cache, the CPU cache may be used by one or more processors 203 to reduce memory latency and access time. A processor 203 may retrieve data from or write data to the CPU cache rather than reading/writing to memory 215, which may improve the speed of these operations. In some examples, a database cache may be created in which certain data from a database 221 is cached in a separate smaller database in a memory separate from the database, such as in RAM 205 or on a separate computing device. For instance, in a multi-tiered application, a database cache on an application server may reduce data retrieval and data manipulation time by not needing to communicate over a network with a back-end database server. These types of caches and others may be included in various embodiments, and may provide potential advantages in certain implementations of devices, systems, and methods described herein, such as faster response times and less dependence on network conditions when transmitting and receiving data.

Although various components of computing device 200 are described separately, functionality of the various components may be combined and/or performed by a single component and/or multiple computing devices in communication without departing from the disclosure.

### Example Machine Learning Models

As described above, various machine learning models may be configured to determine characteristics associated with a fermentation process. Turning now to FIG. 3, a conceptual diagram of such a machine learning model is illustrated. The example of FIG. 3 illustrates just one example configuration of a deep learning model 300 that may be configured to generate output 312, which may indicate a fermentation state. Model 300 may determine the indication of such a fermentation state based on input 301.

At a high level, model 300 may comprise layers, such as an input layer 302, one or more hidden layers, such as LSTM layers 304, 306, 308, and output layer 310. Input layer 302 may be configured to receive input 301. Input layer 302 may forward the received input to a first hidden layer, such as LSTM layer 304. Each hidden layer may, in turn, receive an output from a previous hidden layer, and generate and forward an output to a subsequent hidden layer. For example, LSTM layer 306 may receive input from LSTM layer 304, and may generate one or more outputs, and forward those outputs to LSTM layer 306. This process may proceed until the last hidden layer (LSTM layer 308 in the example of FIG.3) generates output and forward that output to output layer 310. Output layer 310 may generate output 312, which may indicate a fermentation state that associated with a fermentation process. While model 300 is illustrated as having a given architecture comprising various layers, it should be understood that more or fewer layers may be included in model 300. For example, there may be additional layers present in model 300, such as RNN layers, convolutional layers, recurrent neural network layers, such as a Gated Recurrent Units (“GRUs”) or LSTM layers, dense/fully connected layers, and/or various other layers.

LSTM layer 304 may comprise one or more LSTM units, such as LSTM units 304a-304n. At a high level, each LSTM unit may be configured to process a sequence of data, remember values over a given timer interval, and output values based on an activation function. In the illustrated example, LSTM unit 304a may be a first LSTM unit that outputs to LSTM layer 304b and also outputs to LSTM unit 306a. In this manner, LSTM unit 304a may represent a first state, and LSTM unit 304b may represent a second (e.g., future) state. Each LSTM unit within a given layer, such as LSTM layer 304 may be bidirectionally connected to each neighboring LSTM unit that layer. For example, LSTM layer 304b may be bidirectionally connected to LSTM unit 304a and another LSTM layer (not shown) within LSTM layer 304. Such bidirectional connections may allow each LSTM layer to receive previous LSTM state as well as future LSTM state.

Each LSTM unit in a given layer may be configured to generate output and forward it to a subsequent layer. For example, the LSTM units of LSTM layer 304 may generate output and forward it to LSTM layer 306. In turn, LSTM layer 306 may generate and forward the output from each of its LSTM units 306a-306n to a subsequent LSTM layer. After passing through all hidden layers of model 300, the final hidden layer, in this example LSTM layer 308, may forward output from each of its LSTM units to output layer 310.

As explained above, models, including model 300 may have hyperparameters that define aspects of the model that are not trained. According to an embodiment, such hyperparameters for model 300 comprise four bidirectional LSTM layers. The first, second, third, and fourth LSTM layers may have 128, 320, 192, and 64 units, respectively.

Output layer 310 may comprise a fully connected/dense layer that may be configured to generate and output probabilities for one or more fermentation states. In some examples, output layer 310 may be configured to execute a logistic function, such as a softmax function, that may generate a vector of respective probabilities for each fermentation state based on the output from the preceding layer in model 300.

In a simplified example in which there are only six fermentation states, the respective probabilities of each of these states may be represented by the vector {0.01, 0.03, 0.07, 0.8, 0.05, 0.04}, in which all probabilities in the vector have a sum equal to one. According to such an example, the fourth value represented in the vector has the highest probability. This may correspond to the model determining that the known state, “fermentation performance decline,” has the highest probability of all possible fermentation states.

Input 301 may comprise one or more input values. In some examples, input 301 may indicate parameters associated with a fermentation process. Such variables may be time-series data over a historical time window. In an embodiment, such parameters may take the form of variables that may represent features that may be analyzed by a machine learning model. At a high level, such parameters may be categorized into: (1) process inputs, (2) metabolite and biocatalyst concentrations, (3) inline measurements of pH, and/or oxidation-reduction potential (ORP), (4) operating conditions, such as agitation and biocatalyst dilution rate, (5) derived values, (6) process outputs, and/or (7) fermentation setup information, such as a type of microbe and/or metal(s) present in the fermentation. Such input values may provide information on conditions associated with a fermentation process such as: temperature, mass transfer, hydrodynamics, cellular/biocatalyst metabolism, heterogeneity of culture, and/or nutrient availability as just some examples. Such inputs may take various other forms as well. These inputs are explained in greater detail below.

Such metabolite and biocatalyst concentrations may include indications, such as biocatalyst concentration as measured by optical density, which may be measured in grams per liter (g/L). In some cases such biocatalyst concentrations may be represented as capacitance viable biomass. Additionally or alternatively, such metabolite concentrations may include concentrations of metabolites such as: phosphate, acetate, ethanol, lactate, and/or (2,3-butanediol) (BDO). Such metabolite concentrations may be measured online (more frequently) or offline (less frequently) by high performance liquid chromatography (HLPC), and may be measured in g/L. In some cases, the delta between online and offline metabolite concentrations may be input to the model, which may be further informative to the model (e.g., model 300) of the fermentation process. For example, in some instances, such online and offline metabolites concentrations may diverge

Process inputs may take various forms. According to some examples, such process inputs may generally be related to gasses that may be input to a bioreactor. Such input gasses inputs may include input gas percentages of CO, CO₂, H₂, and/or N₂, as some non-limiting examples. Such input gas percentages may indicate a percentage of overall gas that is coming into the bioreactor. As an example, if a variable corresponding to input CO₂ is equal to 43%, this may indicate that 43% of all gas that is coming into the bioreactor is carbon dioxide. Such process inputs may also include an input gas flow rate. Such an input gas flow rate may be measured in kilograms per hour (kg/hour), according to some examples. Such process inputs may take various other forms as well.

Process outputs may take various forms, but may generally be related to gasses that may be output (e.g., exit) from a bioreactor. Examples of such process inputs may include input gas percentages of CO, CO₂, H₂, H₂5, and/or N₂, as some non-limiting examples. Such output gas percentages may indicate a percentage of overall gas that may be coming out the bioreactor. As an example, if a variable corresponding to output CO is equal to 43%, this may indicate that 43% of all gas that exiting a bioreactor is carbon monoxide. Such process outputs may take various other forms as well.

In terms of inline pH and ORP measurements, both pH and ORP may be measured by an online probe. ORP may be measured in millivolts according to some examples.

Operating conditions that may be input to a model may comprise agitation as just one possible example. For a CSTR bioreactor, agitation may be measured in revolutions per minute (RPM), or in other units for non-CSTR bioreactors. Biocatalyst dilution rates may be comprised of inlet and output dilution rates. Such dilution rates may indicate the liquid input to, and output from a bioreactor. Biocatalyst dilution relates may also include one or more solid dilution rates, which may indicate an amount of a biocatalyst-containing media stream that leaves (e.g., exits) the reactor. Such dilution rates may be measured as an amount of dilution per day. Such dilution rates may take various other forms as well.

At a high level, derived input values are values that may be calculated based on other input values. Providing such derived values as input to a model may improve the accuracy of such a model in some cases. Examples of some such derived values may include CO, H₂, and/or CO₂ uptake rates, which may be calculated based on other input values described above. Additionally or alternatively, such derived values may include input and/or output gas flow rates. Such flow rates may be represented in units of cubic meters per hour, in one possible example. There may be numerous other such derived values that may be calculated and input to models described herein as well.

Such fermentation setup information may include indications of parameters such as temperature of a bioreactor, and/or liquid volume in a bioreactor. Additionally, or alternatively, such setup information may include values that indicate a strain of microbe being used in the fermentation process, metals, vitamins, and/or online separation type, which may be one-hot encoded in some examples. Online separation type may indicate whether a bioreactor has cell recycling, vacuum distillation, and/or a centrifuge (e.g., parameters related to design of a bioreactor). Fermentation setup information may take various other forms as well.

Input 301 may undergo preprocessing before being input to model 300. For example, a computing device (e.g., computing device 140) may downsample such input data from a higher time resolution to a lower time resolution representation, for instance hourly (e.g., from multiple data points per hour to one data point per input value per hour). Additionally or alternatively, input 301 may be padded, and/or normalized before entering model 300. For example, input 301 may be z-score normalized before being forwarded into model 300.

Input 301 may thus represent multiple variables at each of a plurality of time steps. For example, input 301 may indicate values for a given set of input variables for each hour of a twelve hour window.

After input 301 has passed through all the hidden layers of model 300, output layer 310 may output a fermentation state for each time step. In some examples, output layer 310 may generate a set of probability values for each time step. Each set of probability value may indicate a probability, as determined by model 300, that a respective time step is in that fermentation. Each respective probability value for each fermentation state may be represented by a respective floating point value in a vector comprised of floating point numbers. There may be, for instance, thirteen possible fermentation states, as just one example. These states may include: (1) “Early start-up,” (2) “Late start-up,” (3) “Adjustment,” (4) “Stable state,” (5) “Foaming or level change,” (6), “Process upset—loss of base dosing,” (7) “Process upset—gas blend disruption,” (8) “Fermentation performance decline,” and/or (9) “Fermentation performance improvement,” as just some examples. There may be various other such fermentation states as well.

As an example, input data for 12 time steps, corresponding to a 12-hour time window, may be input to model 300. For each time step (e.g., an hour), there may be an input value for some, or all of the variables described above. This input data (e.g., input 301) flows first through input layer 302, and then through the hidden layers, LSTM layers 302, 304, 306, 308, etc., and finally through output layer 310. Each layer may transform values received from the previous layer. Output layer 310 may calculate an output value for each hour-long timestep. Thus, in accordance with this example, there may be 12 output values, one for each hour. Each such output value may comprise an output vector that indicates fermentation state probabilities during that time window for each known fermentation state.

By inputting a recently-captured window of fermentation data, model 300 may determine a current fermentation state of the bioreactor. For example, if a time window from each of the previous three hours is input to model 300, the known fermentation state with the highest probability that is associated with the most recent time window may indicate a current fermentation state for the bioreactor.

It should be understood that model 300 is just one example machine learning model architecture, and that model 300 may take various other forms as well.

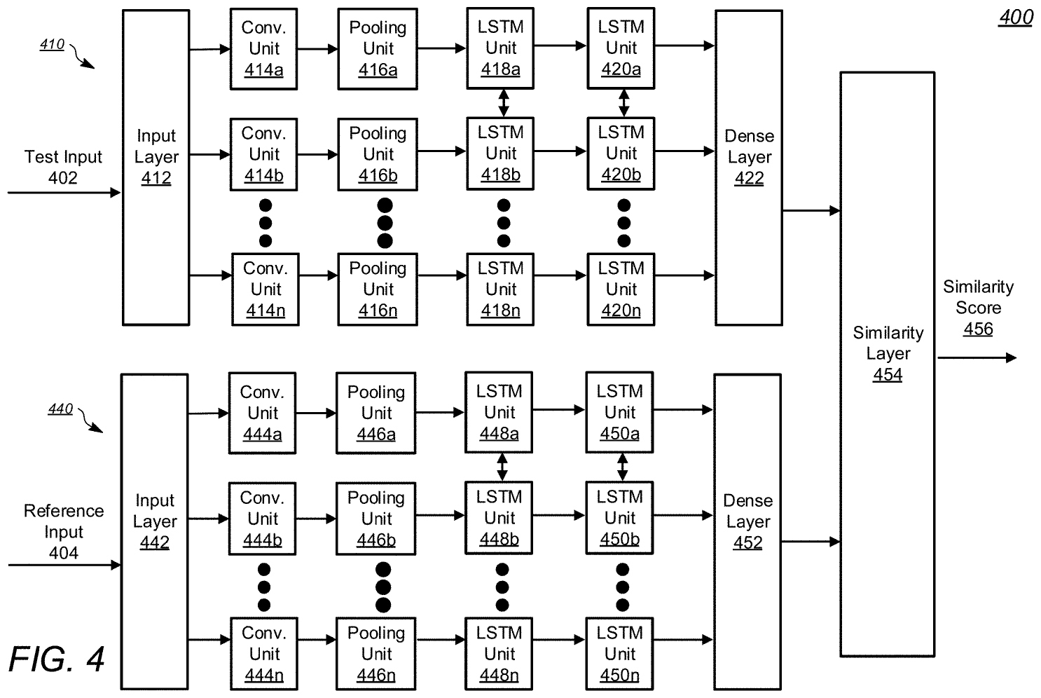

As described above, a machine learning model may also be configured to use similarity learning techniques to determine a fermentation state, which may be associated with a fermentation process of a bioreactor. An example of such a similarity learning model is shown in FIG. 4 as model 400. In the example of FIG. 4, model 400 includes two identical networks, 410 and 440. That is, networks 410 and 440 may have identical weights and hyperparameters. At a high level, model 400 may compare two inputs to determine how similar the two inputs are to each other. According to some examples, the two inputs may comprise data associated with a known fermentation state, and data associated with a fermentation process having an unknown fermentation state. A computing device may assign a known fermentation state to the unknown fermentation state by determining a known fermentation state that is most similar to the unknown fermentation state.

Using such similarity learning techniques may provide various advantages. For instance, such similarity learning networks require fewer training examples of specific fermentation states as compared to other types of neural networks. Further, such similarity learning neural networks can be used to identify fermentation states that have not yet been described and/or characterized by fermentation scientists. Still further, such similarity learning networks may be capable of identifying fermentation states that are difficult for fermentation scientists to identify.

Model 400 may receive test input 402 and reference input 404. Test input 402 may comprise fermentation data that may be associated with an unknown fermentation state, and reference input 404 may comprise data associated with a known fermentation state. Test input 402 may be forwarded into network 410, and input 404 may be forwarded into network 440. Network 410 may be comprised of hidden layers 414, 416, 418, and 420, and network 440 may be comprised of hidden layers 444, 446, 448, and 450. Networks 410 and 440 may further include dense/fully connected layers 422, 452, respectively. The output from dense layers 422, 452 may be forwarded to similarity layer 454, which may be configured to execute a similarity function based on the inputs received from dense layers 422 and 452. Such a similarity function may be configured to generate a numeric representation of a similarity between test input 402 and reference input 404 as transformed by the layers of networks 410 and 440.

Model 400 may be trained to determine whether inputs 402 and 404 are similar during a training phase. During such a training phase, ground truth data associated with known fermentation states may be input to models 410, 440. For example, model 400 may be trained by inputting data from two known similar fermentation states, or two known dissimilar fermentation states into model 400. Model 400 may output a numeric representation of a similarity between the two inputs. Based on whether the numeric similarity output by model 400 for two inputs is correct, the weights and/or similarity function of model 400 may be adjusted to produce more accurate predictions based on minimizing a cost function and/or backpropagation. Such a cost function may be based on mean-squared error in some examples. In some examples, such a cost function may be minimized by algorithms such as gradient descent, stochastic gradient descent, or the Adam optimization algorithm, etc. A cost function may be minimized (e.g., optimized) in various other manners as well. According to some examples, model 400 may be configured to perform gradient clipping when optimizing such a cost function. Gradient clipping may force gradient values not to exceed a specific maximum or minimum value, which may improve model accuracy.

After undergoing sufficient training, model 400 may be configured to predict whether test input 402 and reference input 404 are similar or different fermentation states. Such predictions may be made during an execution phase. During such an execution phase, test input 402 may comprise fermentation data associated with an unknown fermentation state, and reference input 404 may comprise fermentation data associated with a known fermentation state. By comparing test input 402 against reference input 404, similarity model 400 may be used to compare known fermentation state data against unknown fermentation state data to determine a state of a fermentation process. For instance, a computing device (e.g., computing device 140) may store well-defined examples for each possible fermentation state in a data store, such as a database. To determine the fermentation state of test input 402, computing device 140 may iteratively input example data associated with each known fermentation state as reference input 404 to compare against test input 402. Model 400 may compare reference input 404 for that state against unknown input 402 to determine a similarity score between test input 402, associated with the unknown fermentation state, and reference input 404, associated with the known fermentation state. This process of comparing known fermentation state data against unknown fermentation state data may be repeated until all known fermentation states have been compared to unknown input 402. Computing device 140 may determine that the fermentation state corresponding to test input 402 is the known fermentation state that has the highest similarity score with test input 402.

The input data that may be used to train and execute model 400 may be similar to the input data described in connection with FIG. 3, above. For example, such input data may be time-series data comprising features related to the fermentation process, such as input and output gas flow, strain, agitation, intended pH, temperature, etc. Each input data value may represent a value of a feature over a given window of time, such as an hour as one example. In such an example, for each hour, there may be an input value for each input feature/variable (e.g., pH, temperature, etc.).

The data that is input to model 400 may undergo preprocessing similar to the preprocessing described in connection with model 300. For example, the input data may undergo preprocessing such as downsampling, padding, and/or normalization. The input data may undergo various other forms of preprocessing as well.

While model 400 is illustrated as being comprised of an input layer, a convolutional layer, a pooling layer, LSTM layers, and an output layer, it should be understood that model 400 may take various other forms as well. It should be understood model 400 may have more or fewer layers, which may be of the same or different types as the layers shown in FIG. 4.

According to an embodiment, networks 410, 440 may each have four hidden layers. In the example of FIG. 4, networks 410, 440 may have a first, convolutional layer having 32 hidden units, and a filter size of five. The convolutional layer in networks 410, 420 may be connected to a max pooling layer (416, 446), which may have size of five units and a stride of two. The max pooling layer of networks 410, 420 may be connected to a first bidirectional LSTM layer (418, 448) having 32 hidden units, which in turn, may be connected to a second bidirectional LSTM layer (420, 450) have 16 hidden units. The second LSTM layer may be connected to a dense/fully connected layer (422, 452), which may in turn be connected to similarity layer 454, as shown in FIG. 4. Model 400 may take various other forms as well. According to some examples the LSTM layers of model 400 may have a dropout rate of 50% (0.5) during a training phase. According to some other examples, the training phase dropout rate may be 20% (0.2). The training phase dropout rates of such LSTM layers may take various other values as well.

As mentioned above, similarity layer 454 may be configured to execute a similarity function to output similarity score 456. Similarity score 456 may be a value that indicates a similarity between test input 402 and reference input 404 as determined by model 400. According to some examples, the similarity function may take two embeddings as inputs, one embedding generated by the last layer of network 410, and another embedding generated by the last layer of network 440. Such embeddings may each represent features of respective fermentation states in a multi-dimensional space, for example, a 128-dimensional space, as one possible example. The multi-dimensional space may be represented by a floating point value for each dimension (e.g., for a 128-dimensional space, the space may be represented by 128 floating point values). The similarity function of similarity layer 454 may perform logistic regression on the two embeddings to determine similarity score 456. In some examples, similarity layer 456 may determine the element-wise differences (e.g., absolute values) between the two embeddings. These element-wise differences may be used as inputs to a logistic regression executed by similarity layer 454 that outputs similarity score 456. Such a logistic regression function may comprise a binary classification algorithm according to some examples.

As also mentioned above, to determine the state that is most similar to an unknown fermentation state, test input 402 may be iteratively compared against data from known fermentation states. However, given that the same known fermentation states, and therefore the same embeddings corresponding to those fermentation states may be repeatedly compared to test input 402 associated with an unknown state, such embeddings may not be re-computed for each comparison. Rather, the embeddings for all known states may be pre-computed and compared to the embedding that network 410 generates for the unknown state. In this manner, model 400 may be optimized to avoid re-computation by network 440 of embeddings of known fermentation states, which may improve the execution performance of model 400.

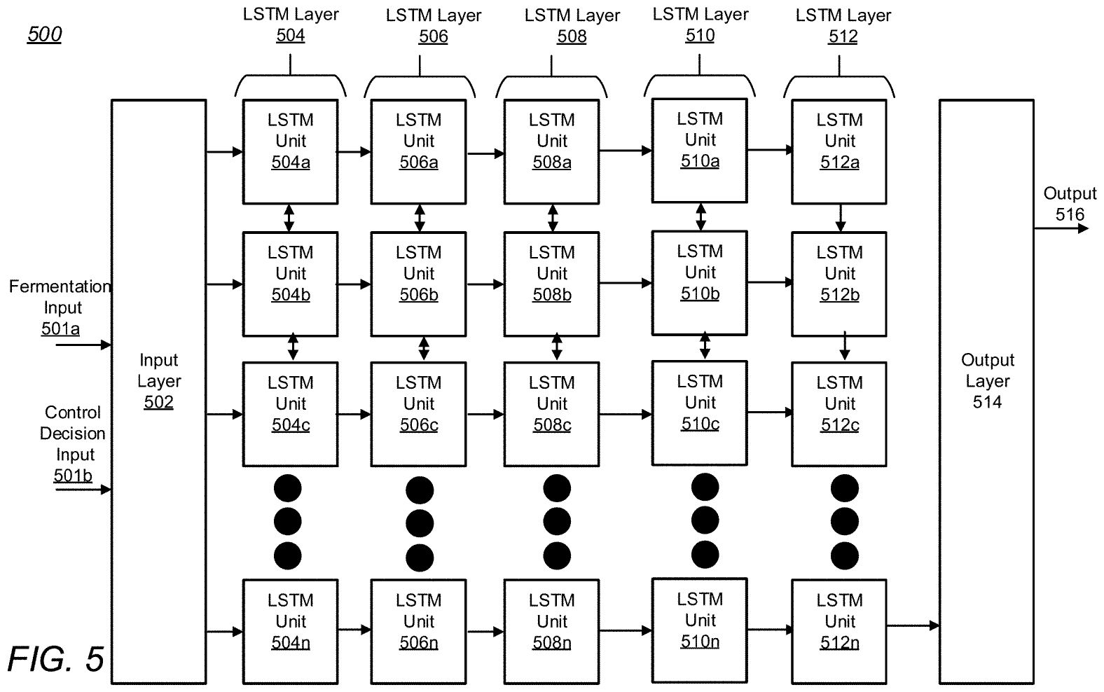

As described above, a machine learning model may be configured to predict future characteristics associated with a fermentation process. An example of such a model is shown in FIG. 5. FIG. 5 shows an example deep learning model 500 for predicting a future characteristics associated with a fermentation process. At a high level, model 500 may receive historical fermentation data (e.g., time-series historical fermentation data), and a time window of future control decisions as input, and may output predictions of metabolite production for the time window of control decisions based on such inputs. As an example, model 500 may receive historical fermentation data, such as the input data described above with respect to FIGS. 3 and 4. Model 500 may also receive future control decisions, such as gas flow rate, agitation, temperature, water/nutrient flow rate, and/or pH level over a future 48-hour window of time. Based on these inputs, model 500 may output a prediction of metabolites, such as acetate and/or ethanol as some non-limiting examples.

As described above, model 500 may be trained to minimize a cost function based on training data that may be input to model 500 during a training phase. The training data for model 500 may comprise variables associated with a fermentation process, such as agitation, strain, metals, pH level, etc., such as those described above with respect to models 300 and 400. Such variables may be downsampled to a specific resolution (e.g., hourly resolution), and/or normalized (e.g., z-score normalized) as described above. Additionally, outlier values may be removed from the input data using various techniques known in the art. Such historical input data may be represented over a historical time window, such as 14 days in the past as one example. The training data used to train model 500 may comprise a window of ground truth control decisions that occurred in a window (e.g., a 48-hour window) after a corresponding historical window of fermentation data. Such training data may further comprise ground truth metabolite production that occurred over the control decision time window (e.g., 48 hours).

As with other models described herein, model 500 may be configured to perform various functions during training to avoid overfitting. As some examples, model 500 (e.g., model 500 executing on computing device 140) may be configured to perform dropout regularization. Additionally or alternatively, model 500 may be configured to perform gradient clipping during the training phase to avoid vanishing or exploding gradients, which may slow down or stop training of such a model.

After model 500 has undergone sufficient training, model 500 may be executed, as part of an execution phase. During such an execution phase, model 500 may make predictions, based on historical fermentation data and future control decisions, of metabolite production that may result from those control decisions over a future time window (e.g., a 48-hour time window, as one possibility).

The predictions that are output by model 500 for a given window of future control decisions for a fermentation process may provide various insights into the future of that fermentation process. As an example, such predictions may provide indirect guidance, which may provide an indication to fermentation personnel that an adverse event associated with a fermentation process is likely to occur in the future. As another example, such predictions may be used to provide direct guidance. Such direct guidance may provide suggestions to fermentation personnel to maintain or obtain a desired fermentation state. Such direct guidance may specify process controls (e.g., gas flow rate, agitation, temperature, water/nutrient flow rate, pH, etc.) that may be used to obtain the desired fermentation state.

Actual historical metabolite output and control decisions that were observed over the window of control decisions may later be used as additional training data to further train model 500. Using such observed historical fermentation data as additional training data may further improve the accuracy of model 500. Using such data to train model 500 may be a form of self-learning. Such self-learning may take various forms. As one example, a window of actual control decisions over a time window, such as a 48-hour window, and actual metabolite (e.g., acetate and/or ethanol) production over that time window, along with a window of historical fermentation data, may be used as training data to further train model 500. Such self-training may take various other forms as well.

Architecturally, model 500 may comprise an input layer 502, one or more hidden layers, such as LSTM layers 504, 506, 508, 510, 512, and an output layer 514, according to some examples. Input layer 502 may be configured to receive input fermentation input data 501a and control decision input 501b. As explained above, fermentation input 501a may comprise variables associated with a fermentation state over a historical window of time (14 days as one example). Control decision input 501b may comprise future control decisions over a future time window (e.g., 48 hours).

Input layer 502 may forward the received inputs 501a, 501b to a first hidden layer, such as LSTM layer 504. Each hidden layer may, in turn, receive an output from a previous hidden layer, and generate and forward an output to a subsequent hidden layer. For example, LSTM layer 506 may receive input from LSTM layer 504, and may generate one or more outputs, and forward those outputs to LSTM layer 506. This process may proceed until the last hidden layer, LSTM layer 512 generates its output and forward that output to output layer 514. Output layer 514 may generate output 516. Output 516 may indicate predicted metabolite production over the future time window (for example a 48 hour time window). While model 500 is illustrated as having five hidden LSTM layers, it should be understood that more or fewer layers may be included in model 500. For example, there may be additional layers present in model 500, such as convolutional layers, recurrent neural network layers, such as a Gated Recurrent Units (“GRUs”), dense/fully connected layers, and/or various other layers as well.

As explained above, models, including model 500 may have hyperparameters that define aspects of the model that are not trained. According to the embodiment shown in FIG. 5, model 500 may comprise five LSTM layers. The first, second, third, fourth, and fifth LSTM layers may have 128, 352, 192, 384, and 32 units, respectively. The first through fourth LSTM layers may be bi-directional LSTM layers, and the final LSTM layer 512 may be unidirectional according to some examples. Output layer 514 may comprise a dense/fully connected layer, which may have dimensions 48×2. As an example output layer 514 may comprise two outputs, which may correspond to predicted values of two metabolites of a fermentation process. Such metabolites may comprise acetate and ethanol according to some examples. Output layer 514 may output 48 values per output metabolite, one value corresponding to each hour of the future control decision time window.

According to another embodiment, model 500 may comprise an architecture other than that shown in FIG. 5. For example, model 500 may be comprised of input layer 502, a CNN layer, a first LSTM layer, and a second LSTM layer, which may be connected to an output layer, such as output layer 514. Input layer 502 may be connected to the CNN layer, which may be connected to the first LSTM layer. According to such an embodiment, the first LSTM layer may have 64 units, and the second LSTM may have 256 units.

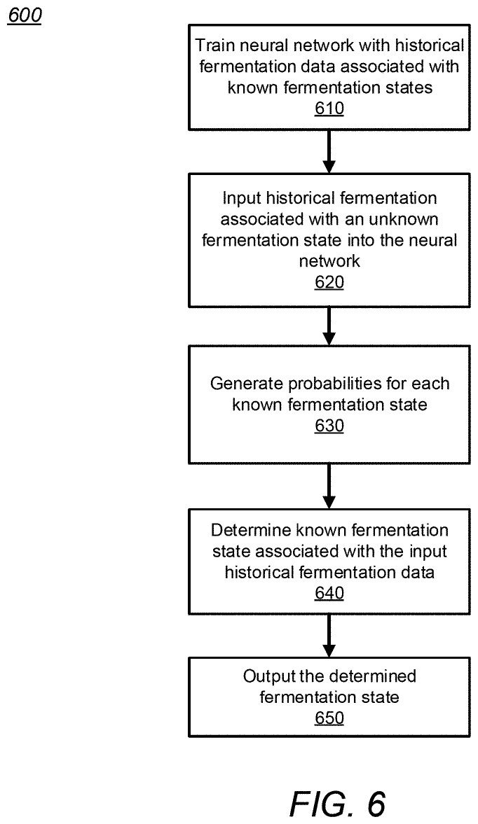

As discussed above, various models may be configured to determine a fermentation state that is associated with a fermentation process of a bioreactor. FIG. 6 illustrates an example flow chart of a process for configuring a model to determine such a fermentation state. Some or all of the steps of process 600 may be performed using one or more computing devices as described herein. In a variety of embodiments, some or all of the steps described below may be combined and/or divided into sub-steps as appropriate.

At step 610, a computing device (e.g., computing device 140) may be configured to train a model, such as model 300 (illustrated in FIG. 3) comprising a deep learning neural network. Such training may occur during a training phase. During such a training phase, historical fermentation data may be input into the deep learning neural network. The training data may also include known fermentation states that are associated with the historical fermentation data. For example, a first known fermentation state may be associated with the first set of historical data, and a second known fermentation state may be associated with the second set of historical data. Based on the training data, a computing device (e.g., the model executing on computing device 140) may minimize a cost function, in order to define a classifier that determines a probability for each possible fermentation state. In some examples. Such a cost function may be minimized using various forms of gradient descent or Adam optimizer, as some examples. The model may also use backpropagation to adjust weights of the neural network. The step of inputting such training data, adjusting the similarity function, and adjusting weights via backpropagation may be repeated until the model reaches a satisfactory prediction accuracy, or another condition has been met.

At step 620, the computing device may input historical fermentation data into the first deep learning neural network. The historical fermentation data may be associated with an unknown fermentation state of a fermentation process of a bioreactor, and may be time-series data. As described above, such historical time-series fermentation data may be comprised of process inputs; metabolite and/or biocatalyst concentrations; inline measurements of pH, ORP, and/or temperature; operation conditions such as agitation and/or biocatalyst dilution rate; derived values; and fermentation setup information. In some examples, such historical time-series input data may be preprocessed before being input to the first and second models. For example, such historical time-series input data may be down-sampled to an hourly time resolution, padded, normalized (e.g., z-score normalized), etc. Such historical time-series data may be measured over a window of time (e.g., a window of 14 days, etc.).

At step 630, the deep learning neural network may generate probabilities for each of the known fermentation states. Each probability may a likelihood that the input historical fermentation data belongs to that known fermentation state. To generate these probabilities, the computing device may cause the input historical fermentation data to pass through each layer of the deep learning neural network. Each layer of the neural network may receive and transform input received from a previous layer of the neural network. According to an example, the neural network may have at least one RNN layer, and a final dense/connected (output) layer. According to some examples, the neural network may be comprised of four bidirectional LSTM layers. The first layer may have 128 units, the second layer may have 320 units, the third layer may have 192 units, and the fourth layer may have 64 units, as an example. The final layer of the neural network may be an output layer, which may be configured to output a probability for each known fermentation state. Each probability may indicate a likelihood that the input historical fermentation data are associated with a corresponding known fermentation state. In some examples, the output layer may be configured to execute a softmax function to generate such probabilities for each known fermentation state.

At step 640, the computing device may determine a known fermentation state that is associated with the input historical fermentation data. The computing device may determine such a known fermentation state by identifying the known fermentation state having the highest probability. The known state having the highest probability may then be selected as the known fermentation state that is associated with the input historical fermentation data.

At step 650 the computing device may output the determined fermentation state. In some examples, the fermentation state may be output to another computing device, such as a monitoring system. According to various examples, the fermentation state may be output to a display screen, which may be at or near a bioreactor associated with the fermentation process. The fermentation state may be output in various other manners as well.

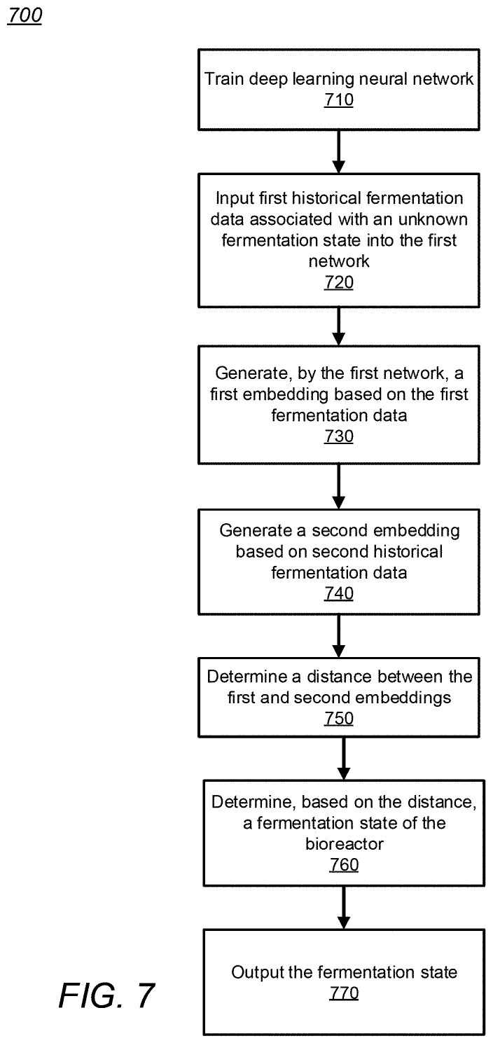

As discussed above, various models may be configured to determine a fermentation state that is associated with a fermentation process of a bioreactor. FIG. 7 illustrates an example shows a flow chart of a process for configuring a model to determine such a fermentation state. Some or all of the steps of process 700 may be performed using one or more computing devices as described herein. In a variety of embodiments, some or all of the steps described below may be combined and/or divided into sub-steps as appropriate.

At step 710, a computing device may train a model, such as model 400 (illustrated in FIG. 4) having first and second identical deep learning networks. Such training may occur during a training phase. During such a training phase, first and second sets of historical fermentation state data may be input into the first and second deep learning neural networks, respectively. The training data may also include known fermentation states. For example, a first known fermentation state may be associated with the first set of historical data, and a second known fermentation state may be associated with the second set of historical data. Based on the training data, a computing device (e.g., the model executing on computing device 140) may optimize a cost function, in order to define a similarity function that indicates a similarity between the first and second sets of historical input data. The model may also use backpropagation to adjust weights of the first and second neural networks in an identical manner. The step of inputting such training data, adjusting the similarity function, and adjusting weight via backpropagation may be repeated until the model reaches a satisfactory prediction accuracy, or another condition has been met (e.g., comparisons of each possible fermentation state have been made).

At step 720, the computing device may input first historical time-series fermentation data into the first deep learning neural network. The first historical time-series fermentation data may be associated with an unknown fermentation state of a fermentation process of a bioreactor. As described above, such historical time-series fermentation data may be comprised of process inputs; metabolite and/or biocatalyst concentrations; inline measurements of pH, ORP, and/or temperature; operation conditions such as agitation and/or biocatalyst dilution rate; derived values; and/or fermentation setup information. In some examples, such historical time-series input data may be preprocessed before being input to the first and second models. For example, such historical time-series input data may be down-sampled to an hourly time resolution, padded, normalized (e.g., z-score normalized), etc. Such historical time-series data may comprise a historical window of time (e.g., a window of 14 days, etc.).

At step 730, the first deep learning neural network may generate a first embedding based on the first historical time-series data. To generate the first embedding, the computing device may cause the first historical time-series data to pass through each layer of the first deep learning neural network. Each layer of the first neural network may receive and transform input received from a previous layer of the neural network. According to an example, the first neural network may have a first convolutional layer, second, third, and fourth bi-directional LSTM layers, and a final dense/connected layer. The final dense/connected layer may generate a first embedding. After passing through the dense/connected layer, the first embedding may be forwarded to a similarity layer.

At step 740, the computing device may generate a second embedding based on second historical time-series fermentation data. Such second time-series fermentation data may be data that is associated with a known fermentation state (e.g., stable state, process upset, fermentation performance improvement, fermentation process decline, etc.). Such a second embedding may be generated in various manners. In some examples, the second embedding may be pre-computed based on the associated known fermentation state. Additionally or alternatively, the second embedding may be generated by inputting the second historical fermentation data into the second deep learning neural network.

At step 750, the computing device may determine a distance between the first embedding and the second embedding. In some examples, such a distance may be calculated based on an element-wise difference between the first and second embeddings. Such a distance may be calculated in various other manners as well.

At step 760, the computing device may determine, based on the distance, a fermentation state of the bioreactor. In some examples, such a fermentation state may be determined by a similarity layer that is configured to execute a similarity function. Such a similarity function may comprise a logistic regression function according to some examples.

At step 770 the computing device may output the determined fermentation state. In some examples, the fermentation state may be output to another computing device, such as a monitoring system. According to various examples, the fermentation state may be output to a display screen, which may be located at or near a bioreactor associated with the fermentation process. The determined fermentation state may be output in various other manners as well.

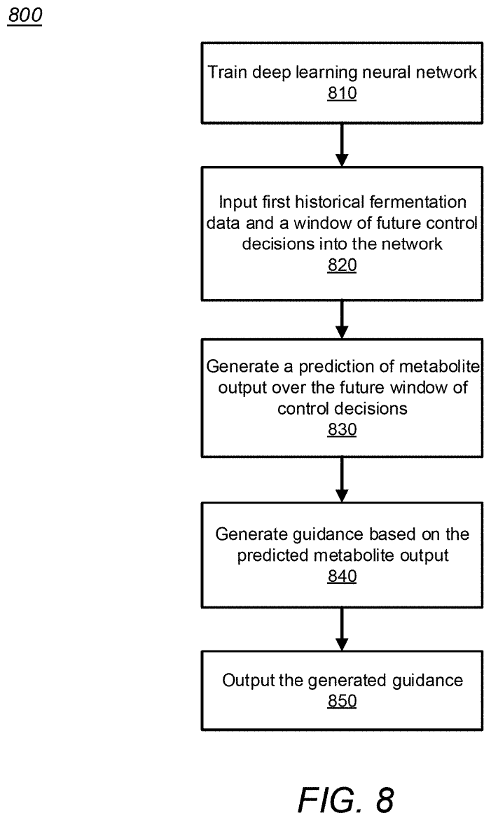

As discussed above, various models may be configured to determine a fermentation state that is associated with a fermentation process of a bioreactor. FIG. 8 illustrates an example flow chart of a process for configuring a model to determine such a fermentation state. Some or all of the steps of process 800 may be performed using one or more computing devices as described herein. In a variety of embodiments, some or all of the steps described below may be combined and/or divided into sub-steps as appropriate.

At step 810, a computing device may train a model, such as model 500 (illustrated in FIG. 5). Such training may occur during a training phase. During such a training phase, first and second sets of historical fermentation state data may be input into the deep learning neural network. The training data may also include a time window of historical fermentation data, a window of control decisions that occurred after the historical time window, and metabolite outputs that were measured over the control decision time window. For example, the historical time window may comprise 14 days of historical fermentation data. The window of control decisions may comprise control decisions such as gas flow rate, agitation, temperature, water/nutrient flow rate, and/or pH, as some examples. The metabolites measured over the window of control decisions may be comprised of acetate and/or ethanol metabolite production as some non-limiting examples. Based on the training data, a computing device (e.g., the model executing on computing device 140) may optimize a cost function, in order to determine a regression function that may predict metabolite production based on historical fermentation data and a window of future control decisions. In some examples, the cost function may be minimized using gradient descent, stochastic gradient descent, or an Adam optimizer function, as some examples. The model may also use backpropagation to adjust weights of the neural network. The step of inputting such training data, adjusting the similarity function, and adjusting weight via backpropagation may be repeated until the model reaches a satisfactory prediction accuracy, or another condition has been met (e.g., comparisons of each possible fermentation state have been made).

At step 820, and during an execution phase, the computing device may input historical fermentation data and a window of future control decisions into the deep learning neural network. Such historical may comprise a historical window of time (e.g., a window of 14 days, etc.) of time-series fermentation data. As described above, such historical time-series fermentation data may be comprised of process inputs; metabolite and/or biocatalyst concentrations; inline measurements of pH, ORP, and/or temperature; operation conditions such as agitation and/or biocatalyst dilution rate; derived values; and fermentation setup information, as some examples. Such a window of future control decisions may comprise a time window (e.g., 48 hours as one possibility) of control decisions as described above in step 810. In some examples, such historical time-series data, and future control decision input data may be preprocessed before being input to the neural network. For example, such historical time-series input data may be down-sampled to an hourly time resolution, padded, normalized (e.g., z-score normalized), etc.

At step 830, and based on the historical fermentation data and future control decisions, the deep learning neural network may generate a prediction of future metabolite production over the control decision time window. To generate the prediction, the computing device may cause the first historical time-series data to pass through each layer of the deep learning neural network. Each layer of the neural network may receive and transform input received from a previous layer of the neural network. According to an example, the neural network may comprise five bi-directional LSTM layers and a final dense/connected output layer. The first, second, third, fourth, and fifth LSTM layers may have 128, 352, 192, 384, and 32 units, respectively. The dense/connected layer may generate and output predictions of each metabolite level over the future window of control decisions (e.g., 48 hours).

At step 840, the computing device may generate guidance based on the predicted metabolite output. Such guidance may take various forms. As an example, such guidance may comprise indirect guidance, which may indicate that there may be an issue or adverse event that may be likely to occur in the future. Additionally or alternatively, such guidance may comprise direct guidance. As an example, such direct guidance may specify corrective action to take to prevent such issues and/or adverse events. As another example, such direct guidance may specify control decisions to maintain a desired fermentation state. Such direct guidance may take various other forms as well.

At step 850 the computing device may output the generated guidance. In some examples, the generated guidance may be output to another computing device, such as a monitoring system. According to various examples, the generated guidance may be output to a display screen, which may be at or near a bioreactor associated with the fermentation process. The generated guidance may be output in various other manners as well.

One or more aspects discussed herein may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML, XML, Python, C/++, R, Java, and/or JavaScript. The computer executable instructions may be stored on a computer readable medium such as a hard disk, optical disk, removable storage media, solid state memory, RAM, etc. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects discussed herein, and such data structures are contemplated within the scope of computer executable instructions and computer-usable data described herein. Various aspects discussed herein may be embodied as a method, a computing device, a data processing system, or a computer program product.

Although the subject matter has been described in language specific to structural features and/or methodological acts, it is to be understood that the subject matter defined in any claim is not necessarily limited to the specific features or acts described above. Rather, the specific features and acts described above

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavor in any country.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (e.g., “or”) should be understood to mean either one, both, or any combination thereof of the alternatives. As used herein, the term “about” means ±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein. Variations of those embodiments may become apparent to those of ordinary skill in the art upon reading the foregoing description. Skilled artisans may employ such variations as appropriate, and it is intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments are described below with the understanding that such embodiments illustrate but not limit the scope of the disclosure and appended claims.

A first embodiment is a computer-implemented method comprising: receiving, from at least one control system associated with a bioreactor, first historical fermentation data associated with an unknown fermentation state, wherein the unknown fermentation state is associated with a fermentation process of a bioreactor; inputting the first historical fermentation data into a first deep learning neural network, wherein a second deep learning neural network is identical to the first deep learning neural network; generating, by the first deep learning neural network and based on the first historical fermentation data associated with the fermentation process, a first embedding; generating, based on second historical fermentation data that are associated with a known fermentation state, a second embedding, wherein: the second embedding is based on the second historical fermentation data that are associated with the known fermentation state; determining a distance between the first embedding and the second embedding; determining, based on the distance, a known fermentation state of the bioreactor; and outputting the determined known fermentation state. Another embodiment is the computer-implemented method of the first embodiment wherein the first deep learning neural network comprises: at least one convolutional layer, at least one pooling layer that is connected to the convolutional layer, and at least one bidirectional recurrent neural network (RNN) layer that is connected to the convolutional layer, the method further comprising: receiving, by the at least one pooling layer of the first deep learning neural network, output from the at least one convolutional layer of the first deep learning neural network; receiving, by the at least one RNN layer of the first deep learning neural network, output from the at least one convolutional layer; outputting, by the at least one RNN layer of the first deep learning neural network, the first embedding; and outputting, by the at least one RNN layer of the second deep learning neural network, the second embedding. Another embodiment is the computer-implemented method of this paragraph wherein the at least one convolutional layer comprises 32 hidden units and has a filter size of five, the pooling layer comprises a max pooling layer, having a size of five, and a stride of two, and the at least one RNN layer comprises: a first bi-directional long short-term memory (LSTM) layer having 32 hidden units, and a second bidirectional LSTM layer having 16 hidden units. Another embodiment is the computer-implemented method of this paragraph wherein the first deep learning neural network comprises: at least one long short-term memory (LSTM) layer; and an output layer that is connected to the at least one LSTM layer, the method further comprising: receiving, by the output layer, output from the at least one LSTM layer, and wherein: the first embedding is output by the output layer of the first deep learning neural network, and wherein the second embedding is output by an output layer of the second deep learning neural network. Another embodiment is the computer-implemented method of this paragraph wherein the distance is an element-wise difference between the first embedding and the second embedding. Another embodiment is the computer-implemented method of this paragraph wherein the first historical fermentation data comprise a first window of time-series data, and the second historical fermentation data comprise a second window of time-series data, the method further comprising: inputting the first window of time-series data into the first deep learning neural network; and inputting the second window of time-series data into the second deep learning neural network. Another embodiment is the computer-implemented method of this paragraph wherein the second historical fermentation data associated with a known fermentation state comprises a plurality of historical time-series data sets, wherein each historical time-series data set of the plurality of historical time-series data sets is associated with a known fermentation state, the method further comprising: inputting each of the plurality of historical time-series data sets into the second deep learning neural network; determining, based on the plurality of historical time-series data sets, an embedding for each known fermentation state, wherein determining the known fermentation state of the bioreactor comprises: determining, based on the embedding for each known fermentation state, a known fermentation state that has a smallest distance from the first embedding. Another embodiment is the computer-implemented method of this paragraph wherein the known fermentation state comprises one or more indication of: a stable state, a fermentation performance improvement, a fermentation performance decline, or a fermentation process upset. Another embodiment is the computer-implemented method of this paragraph further comprising training the first deep learning neural network and the second deep learning neural network based on one or more of: historical fermentation data, fermentation state data, or synthetic fermentation data. Another embodiment is computing device comprising at least one processor; and memory storing instructions that, when executed by the one or more processors, cause the computing device to perform any combination of the methods of this paragraph. Another embodiment is a system comprising: a bioreactor; and a computing device that is coupled to at least one input and at least one output of the bioreactor, wherein the computing device comprises: at least one processor; and memory storing instructions that, when executed by the one or more processors, cause the computing device to perform any combination of the methods of this paragraph. Another embodiment is a non-transitory computer-readable medium comprising instructions that, when executed by one or more processors, cause a computing device to perform any combination of the methods of this paragraph.

A second embodiment is a computer-implemented method comprising: receiving, from at least one control system associated with a bioreactor, a window of historical fermentation data associated with a fermentation process of the bioreactor; receiving a window of future control decisions; inputting the historical fermentation data and the window of future control decisions into a deep learning neural network; predicting, by the deep learning neural network and based on the received window of historical fermentation data and the window of future control decisions, future metabolite production of the fermentation process, wherein the deep learning neural network comprises at least one bi-directional long short-term memory (LSTM) layer; and outputting an indication of the prediction of future metabolite production of the fermentation process. Another embodiment is the computer-implemented method of this paragraph wherein the metabolite production comprises acetate and ethanol production, the method further comprising: determining, after the future time window, actual ethanol and acetate production of the fermentation process during the window of future control decisions; and training the deep learning neural network based on the actual ethanol and acetate production of the fermentation process during the window of future control decisions. Another embodiment is the computer-implemented method of this paragraph further comprising: training the deep learning neural network with windows of historical time-series data. Another embodiment is the computer-implemented method of this paragraph wherein the future control decisions in the window of future control decisions comprise one or more changes to the fermentation process of: gas flow rate; dilution rate; media flow rate; pressure; or agitation. Another embodiment is the computer-implemented method of this paragraph wherein the window of future control decisions comprises no changes to the fermentation process. Another embodiment is the computer-implemented method of this paragraph further comprising: outputting, and for display, an indication of guidance for controlling the fermentation process, wherein the guidance for controlling the fermentation process is based on the window of future control decisions. Another embodiment is the computer-implemented method of this paragraph wherein the guidance comprises at least one of: direct guidance or indirect guidance. Another embodiment a computing device comprising: at least one processor; and memory storing instructions that, when executed by the one or more processors, cause the computing device to perform any combination of the methods of this paragraph. Another embodiment is a system comprising: a bioreactor; and a computing device that is coupled to at least one input and at least one output of the bioreactor, wherein the computing device comprises: at least one processor; and memory storing instructions that, when executed by the one or more processors, cause the computing device to perform any combination of the methods of this paragraph. Another embodiment is a non-transitory computer-readable medium comprising instructions that, when executed by the one or more processors, cause a computing device to: perform any combination of the methods of this paragraph.

A third embodiment is a computer-implemented method comprising: inputting, into a deep learning neural network, historical fermentation data that are associated with an unknown fermentation state; determining, by the deep learning neural network and based on the historical fermentation data, probabilities for each of a plurality of known fermentation states, wherein the deep learning neural network comprises at least one bi-directional long short-term memory (LSTM) layer; determining a known fermentation state having a highest probability based on the probabilities; and assigning the known fermentation state having the highest probability to the unknown fermentation state. Another embodiment is the computer-implemented method of this paragraph wherein the historical fermentation data comprise time-series data. Another embodiment is the computer-implemented method of this paragraph further comprising: training the deep learning neural network to define a regression function based on ground truth historical fermentation data and corresponding known fermentation state data. Another embodiment is the computer-implemented method of this paragraph wherein the at least one LSTM layer is connected to a dense output layer, the method further comprising: outputting, by the dense output layer and based on output generated by the at least one LSTM layer, the probabilities for each of the plurality of known fermentation states. Another embodiment is the computer-implemented method of this paragraph wherein the at least one LSTM layer comprises four LSTM layers, wherein: a first layer of the four LSTM layers has 128 units, a second layer of the four LSTM layers has 320 units, a third layer of the four LSTM layers has 192 units, and a fourth layer of the four LSTM layers has 64 units. Another embodiment is the computer-implemented method of this paragraph, further comprising: training the deep learning neural network based on one or more of: historical fermentation data, fermentation state data, or synthetic fermentation data. Another embodiment is the computer-implemented method of this paragraph, wherein the known fermentation states comprise one or more indication of: a stable state, a fermentation performance improvement, a fermentation performance decline, or a fermentation process upset. Another embodiment a computing device comprising: at least one processor; and memory storing instructions that, when executed by the one or more processors, cause the computing device to perform any combination of the methods of this paragraph. Another embodiment a system comprising: a bioreactor; and a computing device that is coupled to at least one input and at least one output of the bioreactor, wherein the computing device comprises: at least one processor; and memory storing instructions that, when executed by the one or more processors, cause the computing device to perform any combination of the methods of this paragraph. Another embodiment is a non-transitory computer-readable medium comprising instructions that, when executed by one or more processors, cause a computing device to: perform any combination of the methods of this paragraph.

1. A computer-implemented method comprising:
receiving, from at least one control system associated with a bioreactor, first historical fermentation data associated with an unknown fermentation state, wherein the unknown fermentation state is associated with a fermentation process of a bioreactor;
inputting the first historical fermentation data into a first deep learning neural network, wherein a second deep learning neural network is identical to the first deep learning neural network;
generating, by the first deep learning neural network and based on the first historical fermentation data associated with the fermentation process, a first embedding;
generating, based on second historical fermentation data that are associated with a known fermentation state, a second embedding,
wherein:
the second embedding is based on the second historical fermentation data that are associated with the known fermentation state;
determining a distance between the first embedding and the second embedding;
determining, based on the distance, a known fermentation state of the bioreactor; and
outputting the determined known fermentation state.

2. The computer-implemented method of claim 1,
wherein the first deep learning neural network comprises:
at least one convolutional layer,
at least one pooling layer that is connected to the convolutional layer, and
at least one bidirectional recurrent neural network (RNN) layer that is connected to the convolutional layer, the method further comprising:
receiving, by the at least one pooling layer of the first deep learning neural network, output from the at least one convolutional layer of the first deep learning neural network;
receiving, by the at least one RNN layer of the first deep learning neural network, output from the at least one convolutional layer;
outputting, by the at least one RNN layer of the first deep learning neural network, the first embedding; and
outputting, by the at least one RNN layer of the second deep learning neural network, the second embedding.

3. The computer-implemented method of claim 2, wherein:
the at least one convolutional layer comprises 32 hidden units and has a filter size of five,
the pooling layer comprises a max pooling layer, having a size of five, and a stride of two, and
the at least one RNN layer comprises:
a first bi-directional long short-term memory (LSTM) layer having 32 hidden units, and
a second bidirectional LSTM layer having 16 hidden units.

4. The computer-implemented method of claim 1, wherein the first deep learning neural network comprises:
at least one long short-term memory (LSTM) layer; and
an output layer that is connected to the at least one LSTM layer, the method further comprising:
receiving, by the output layer, output from the at least one LSTM layer, and
wherein:
the first embedding is output by the output layer of the first deep learning neural network, and
wherein the second embedding is output by an output layer of the second deep learning neural network.

5. The computer-implemented method of claim 1, wherein the distance is an element-wise difference between the first embedding and the second embedding.

6. The computer-implemented method of claim 5,
wherein:
the first historical fermentation data comprise a first window of time-series data, and
the second historical fermentation data comprise a second window of time-series data,
the method further comprising:
inputting the first window of time-series data into the first deep learning neural network; and
inputting the second window of time-series data into the second deep learning neural network.

7. The computer-implemented method of claim 1, wherein the second historical fermentation data associated with a known fermentation state comprises a plurality of historical time-series data sets, wherein each historical time-series data set of the plurality of historical time-series data sets is associated with a known fermentation state, the method further comprising:
inputting each of the plurality of historical time-series data sets into the second deep learning neural network;
determining, based on the plurality of historical time-series data sets, an embedding for each known fermentation state,
wherein determining the known fermentation state of the bioreactor comprises:
determining, based on the embedding for each known fermentation state, a known fermentation state that has a smallest distance from the first embedding.

8. The computer-implemented method of claim 1, wherein the known fermentation state comprises one or more indication of:
a stable state,
a fermentation performance improvement,
a fermentation performance decline, or
a fermentation process upset.

9. The computer-implemented method of claim 1, further comprising:
training the first deep learning neural network and the second deep learning neural network based on one or more of:
historical fermentation data,
fermentation state data, or
synthetic fermentation data.

10. A computer-implemented method comprising:
receiving, from at least one control system associated with a bioreactor, a window of historical fermentation data associated with a fermentation process of the bioreactor;
receiving a window of future control decisions;
inputting the historical fermentation data and the window of future control decisions into a deep learning neural network;
predicting, by the deep learning neural network and based on the received window of historical fermentation data and the window of future control decisions, future metabolite production of the fermentation process, wherein the deep learning neural network comprises at least one bi-directional long short-term memory (LSTM) layer; and
outputting an indication of the prediction of future metabolite production of the fermentation process.

11. The computer-implemented method of claim 9, wherein the metabolite production comprises acetate and ethanol production, the method further comprising:
determining, after the future time window, actual ethanol and acetate production of the fermentation process during the window of future control decisions; and
training the deep learning neural network based on the actual ethanol and acetate production of the fermentation process during the window of future control decisions.

12. The computer-implemented method of claim 9, further comprising:
training the deep learning neural network with windows of historical time-series data.

13. The computer-implemented method of claim 9, wherein the future control decisions in the window of future control decisions comprise one or more changes to the fermentation process of:
gas flow rate;
dilution rate;
media flow rate;
pressure; or
agitation.

14. The computer-implemented method of claim 9, wherein the window of future control decisions comprises no changes to the fermentation process.

15. The computer-implemented method of claim 9, further comprising: outputting, and for display, an indication of guidance for controlling the fermentation process,
wherein the guidance for controlling the fermentation process is based on the window of future control decisions.

16. The computer-implemented method of claim 9, wherein the guidance comprises at least one of: direct guidance or indirect guidance.

17. A computer-implemented method comprising:
inputting, into a deep learning neural network, historical fermentation data that are associated with an unknown fermentation state;
determining, by the deep learning neural network and based on the historical fermentation data, probabilities for each of a plurality of known fermentation states, wherein the deep learning neural network comprises at least one bi-directional long short-term memory (LSTM) layer;
determining a known fermentation state having a highest probability based on the probabilities; and
assigning the known fermentation state having the highest probability to the unknown fermentation state.

18. The computer-implemented method of claim 17, wherein the historical fermentation data comprise time-series data.

19. The computer-implemented method of claim 17, further comprising:
training the deep learning neural network to define a regression function based on ground truth historical fermentation data and corresponding known fermentation state data.

20. The computer-implemented method of claim 17, wherein the at least one LSTM layer is connected to a dense output layer, the method further comprising:
outputting, by the dense output layer and based on output generated by the at least one LSTM layer, the probabilities for each of the plurality of known fermentation states.

21. The computer-implemented method of claim 17, wherein the at least one LSTM layer comprises four LSTM layers, wherein:
a first layer of the four LSTM layers has 128 units,
a second layer of the four LSTM layers has 320 units,
a third layer of the four LSTM layers has 192 units, and
a fourth layer of the four LSTM layers has 64 units.

22. The computer-implemented method of claim 17, further comprising:
training the deep learning neural network based on one or more of:
historical fermentation data,
fermentation state data, or
synthetic fermentation data.

23. The computer-implemented method of claim 17, wherein the known fermentation states comprise one or more indication of:
a stable state,
a fermentation performance improvement,
a fermentation performance decline, or
a fermentation process upset.
